(19) United States
(12) Patent Application Publication
(10) Pub. No.: US 2014/0004043 A1
(43) Pub. Date: Jan. 2, 2014

(54) CRYPTOPHANE DERIVATIVES AND METHODS OF USE THEREOF

(71) Applicant: Kevin Holman, McLean, VA (US)

(72) Inventor: Kevin Holman, McLean, VA (US)

(73) Assignee: Georgetown Univeristy, Washington, DC (US)

(21) Appl. No.: 13/878,956

(22) PCT Filed: Oct. 12, 2011
(86) PCT No.: PCT/US11/55993

§ 371 Date: Sep. 23, 2013

Related U.S. Application Data
(60) Provisional application No. 61/392,226, filed on Oct. 12, 2010.

(51) Int. Cl. C07D 493/18

(52) U.S. Cl. CPC C07D 493/18
USPC 424/1.81; 549/208; 424/9.32; 424/1.85

(57) ABSTRACT

The present invention relates to the cryptophane derivatives of formula (I) capable of encapsulating small molecules such as noble gases for biological and environmental use. In particular, the invention relates to cryptophane derivatives with high affinity for xenon, which can be used as biosensors in clinical imaging.

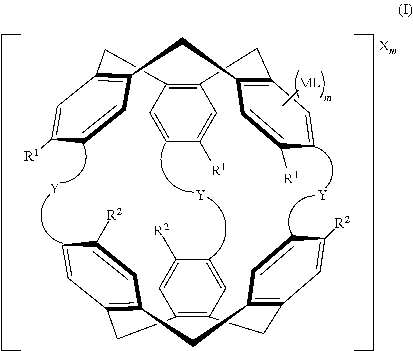

### STATEMENT OF PRIORITY

This application claims priority to U.S. Provisional Application No. 61/392,226, filed Oct. 12, 2010, the disclosure of which is hereby incorporated by reference herein.

### STATEMENT OF GOVERNMENT INTEREST

This invention was made in part with government support under grant number DMR-034916, awarded by the U.S. National Science Foundation. The government has certain rights to this invention.

### INCORPORATION BY REFERENCE

Any foregoing applications and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention

Citation or identification of any document in this application is not an admission that such document is available as prior art to the present invention.

### FIELD OF THE INVENTION

The present invention relates to cryptophane derivatives capable of encapsulating small molecules such as noble gases for biological and environmental use. In particular, the invention relates to cryptophane derivatives with high affinity for xenon, which can be used as biosensors in clinical imaging.

### BACKGROUND OF THE INVENTION

Recently encapsulation of charged or neutral small molecules has been utilized in various practical applications including molecular recognition, drug delivery, separation and storage, biosensing, and catalysis (Brotin, T.; Dutasta, J.-P. Chem. Rev. 2009, 109, 88-130).

The lipophilic cavity of the ball-shaped cryptophane molecules is constructed from two orthocyclophane or cyclotriveratrylene (CTV) moieties connected by three linkers of variable length and constitution. Two main synthetic approaches for the preparation of cryptophanes are the direct (two-step) method and the template method (Brotin, T.; Dutasta, J.-P. Chem. Rev. 2009, 109, 88-130).

An imaging technology based upon transportation of xenon to biological targets via functionalized molecular xenon hosts has recently been proposed (a) Spence, M. M.; Rubin, S. M.; Dimitrov, I. E.; Ruiz, E. J.; Wemmer, D. E.; Pines, A.; Qin Yao, S.; Tian, F.; Schultz, P. G. Proc. Natl. Acad. Sci. USA 2001, 98, 10654-10657. b) Berthault, P.; Huber, G.; Desvaux, H. Prog. NMR Spectrosc. 2009, 55, 35-60) and is supported by proof-of-concept experiments (Schröder, L.; Lowery, T. J.; Hilty, C.; Wemmer, D. E.; Pines, A. Science 2006, 314, 446-449; Wei, Q.; Seward, G. K.; Hill, P. A.; Patton, B.; Dimitrov, I. E.; Kuzma, N. N.; Dmochowski, I. J. J. Am. Chem. Soc. 2006, 128, 13274-13283 ; Roy, V.; Brotin, T.; Dutasta, J.-P.; Charles, M.-H.; Delair, T.; Mallet, F.; Huber, G.; Desvaux, H.; Boulard, Y.; Berthault, P. Chem. Phys. Chem 2007, 8, 2082-2085 ; Schlundt, A.; Kilian, W.; Beyermann, M.; Sticht, J.; Günther, S.; Höpner, S.; Falk, K.; Roetzschke, 0.; Mitschang, L.; Freund, C. Angew. Chem. Int. Ed. 2009, 48,1-5; Brotin, T.; Dutasta, J.-P. Chem. Rev. 2009, 109, 88-130).

For two decades, xenon has received increasing attention as a potent tracer for magnetic resonance imaging (MRI) due to laser-polarized techniques that enhance nuclear polarization and thereby detectability, by several orders of magnitude (Cherubini, A.; Bifone, A. Prog. NMR. Spectrosc. 2003, 42, 1-30).

When hyperpolarized ¹²⁹Xe is inhaled into the lungs or injected in a carrier fluid, it dissolves in the blood and is circulated throughout the body, where it could be imaged in all tissues. However, although the signal enhancement achieved through the use of ¹²⁹Xe is important, it is not sufficient to enable the molecular imaging. Further sensitivity enhancement is needed, and a means is required to obtain specificity to particular molecular or biological targets of interest. To address these issues, Schröder et al. turned to xenon biosensors (Schröder, L.; Lowery, T. J.; Hilty, C.; Wemmer, D. E.; Pines, A. Science 2006, 314, 446-449).

Xenon-based molecular sensors are molecular imaging agents that rely on the exchange of hyperpolarized xenon between the bulk and a specifically targeted host-guest complex. Xenon-based molecular sensors are detectable at concentrations below the threshold of conventional NMR and can reveal important information regarding their local chemical environment.

A few organic synthetic molecular receptors have been reported to bind xenon in solution. The formation of the complexes of xenon with hemicarcerands in organic solution and with cucurbituril host in aqueous solution and with a-cyclodextrin in water (Brotin, T.; Dutasta, J.-P. Chem. Rev. 2009, 109, 88-130) was characterized by the high-field shift of the ¹²⁹Xe NMR signal of the bound xenon compared to the signal of free xenon in solution. The large chemical shift difference is characteristic of a dramatic change in the environment of the xenon guest.

Since the synthesis of cryptophane—A in 1981, considerable progress has been made in designing cryptophanes that exhibit selective encapsulation properties toward organic and inorganic molecules.

It has been already demonstrated that cryptophanes, most notably derivatives of (±)-cryptophane-A (Formula II, R═OCH₃, n=2), are among the best molecular hosts for xenon in organic solution. The stability of the xenon complex and cryptophane-A is characterized by a strong binding constant Kₐ of 3900 at 278K, quite remarkable for the association of a gaseous neutral guest.

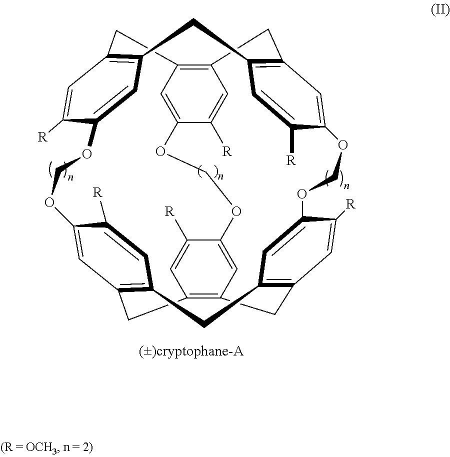

Dmochowski et al. (U.S. Patent Application Publication 2010-0105099) refer to examples of cryptophane-A type compounds. These compounds include tri-triazole propionate cryptophane, crown-saddle (CS)-triallyl cryptophane (TAC) and tri-propargyl cryptophane.

Until recently, cryptophane-A was the best molecular host for xenon in organic solution. One of the most significant problems for biological applications of the xenon-based biosensors arises from poor water solubility of hosts, such as cryptophanes when used under physiological conditions.

The smallest cryptophane core synthesized to date, (±)-cryptophane-1,1,1 (1), was shown to exhibit the largest binding constant with xenon ever measured in an organic solvent (Kₐ˜10,000 M⁻¹ at 293 K in 1,1,2,2-tetrachloroethane-d₂ (TCE-d₂)) (Fogarty et al., J. Am. Chem. Soc. 2007, 129, 10332-10333.).

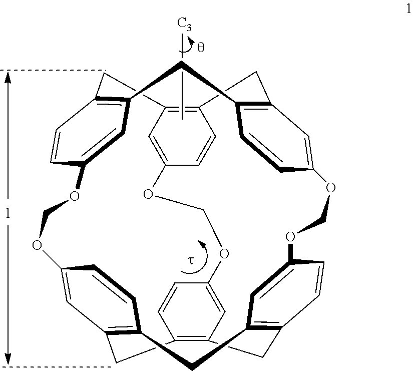

The exceptional xenon binding constant of 1 is largely the consequence of an optimized size match between xenon (V_{Xe}=42 Å³) and the small, spheroidal, arene-lined cavity of the host (V_{c}˜80 Å³). However, while derivatives of 1 are candidates for ¹²⁹Xe—NMR based biosensors, the development of these derivatives of 1 via the attachment of hydrophilic residues has been limited by the lack of modifiable functional groups.

intermediate sized cryptophanes, for example cryptophane-i i2 and 122 have also been synthesized. Preliminary studies indicate that these cryptophanes also encapsulate ¹²⁹Xe. See Kotera et al. Org. Lett. 2011, 13, 2153-2155.

Therefore, a need still exists in the art to develop cryptophane derivatives capable of encapsulating small molecules such as noble gases for biological and environmental use.

### SUMMARY OF THE INVENTION

The present invention relates to metalated cryptophane derivatives of formula (I):

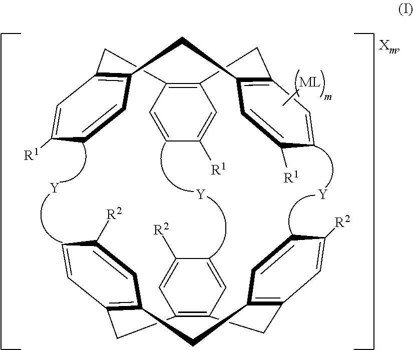

including enantiomers and mixtures of enantiomers thereof, wherein

Y is —OZO—; —CH₂ArCH₂—, —CH₂CH═CHCH₂—, —CH₂C≡CCH₂—,

—OCH₂CH≡CC≡CCH₂O—, —(OC₂CH₂)O(OCH₂CH₂)—;

Z═(CH₂)ₙ;

M is transition metal;

L is ligand;

R¹ and R², independently from each other, are H, (C₁-C₃)alkyl, or (C₁-C₃)alkoxy

X is anionic group;

m is an integer from 1 to 6; and

each n is independently 1 or 2.

Compounds of formula (I) are chiral and are known to exist in enantiomeric forms. Compounds of formula (I) may be a single enantiomer, for example as the (+) or (−) form, or a mixture of enantiomers, for example as a (±) for, which includes, but is not limited to racemic mixtures.

An embodiment of the present invention also includes diastereomers of formula (I). Compounds of formula (I) are known to exist as syn or anti diastereomeric forms, defined by the relative chirality of the two connected cyclotribenzylene units. Another embodiment of the present invention includes the syn diastereomer of formula (I), enatiomers and mixtures thereof.

Incompletely functionalized cryptophanes of formula (I) (m=2-4) will exists in various regioisomeric forms, depending upon the relative positions of the ML substitutuents appended to the arene rings of the cryptophane. An embodiment of the present invention involves any regioisomers or mixtures thereof of crytophanes of formula (I).

Another embodiment of the present invention is directed to a biosensor complex, wherein a small molecule is encapsulated in the cavity of the metalated cryptophane of formula (I).

Another embodiment of the present invention is directed to a biosensor complex of noble gases and cryptophane derivatives of formula (I).

Another embodiment of the present invention is directed to i) a high binding constant (Kₐ) between the noble gas and the cryptophane derivative of formula (I), ii) a large chemical shift difference between bound and free noble gas.

Another embodiment of the present invention is directed to a biosensor complex of Xe and cryptophane derivatives of formula (I).

Another embodiment of the present invention is a method of using xenon encapsulated in cryptophane of formula (I) for molecular imaging.

Another embodiment of the present invention is directed to a method of using the metalated cryptophane of formula (I) for encapsulation of small molecules or atoms, which have to be delivered to the desired biological targets, such as receptors, organs, etc. or removed from the environment as, for example, in case of scavenging pollutants or used to isolate the desired materials from the mixtures or to conduct an isolation or separations process.

Another embodiment of the present invention is directed to a method of using the metalated cryptophane of formula (I) for encapsulation of xenon, which has to be delivered to the desired biological targets, such as receptors, organs, etc. or removed from the environment as, for example, in case of scavenging pollutants or used to isolate the desired materials from the mixtures or to conduct an separations process.

Another embodiment of the present invention is a cryptophane of formula (I) which is water soluble.

Another embodiment of the present invention is a cryptophane of formula (I) which has an empty cavity.

The term “cryptophane” refers to a class of organic supramolecular compounds studied and synthesized primarily for molecular encapsulation and recognition. One possible noteworthy application of cryptophanes is encapsulation and storage of hydrogen gas for potential use in fuel cell automobiles. Cryptophanes can also serve as containers in which organic chemists can carry out reactions that would otherwise be difficult to run under normal conditions. Due to their unique molecular recognition properties, cryptophanes also hold great promise as a potentially new way to study the binding of organic molecules with substrates, particularly as pertaining to biological and biochemical applications. Cryptophane cages are formed by two cup-shaped [1.1.1]ortho cyclophane units connected by three or more bridges (denoted Q in structural formula shown below). There are also choices of the peripheral substitutes Q¹ and Q² attached to the aromatic rings of the units. Most cryptophanes exhibit two diastereomeric forms (syn and anti), distinguished by their symmetry type. This general scheme offers a variety of choices (Q, Q¹, Q², and symmetry type) by which the shape, the volume, and the chemical properties of the generally hydrophobic pocket inside the cage can be modified, making cryptophanes suitable for encapsulating many types of small molecules and even chemical reactions.

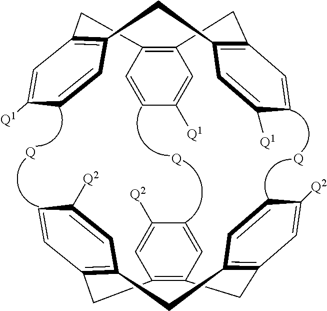

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right and hereby disclose a disclaimer of any previously described product, method of making the product or process of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of and “consists essentially of have the meaning ascribed to them in U.S. Patent law, e.g., they allow for elements not explicitly recited, but exclude elements that are found in the prior art or that affect a basic or novel characteristic of the invention.

These and other embodiments are disclosed or are apparent from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 shows thermal ellipsoid plots of a) 1 from the X-ray crystal structure of 1@0.75H₂O.2CHCl₃, b) the minimally contracted, empty [2]⁶⁺ host as revealed from the X-ray crystal structure of [2][CF₃SO₃]₆.xNO₂Me. The water-occupied (65 Å³) and empty (31 Å³) host cavities, respectively, are depicted in orange.

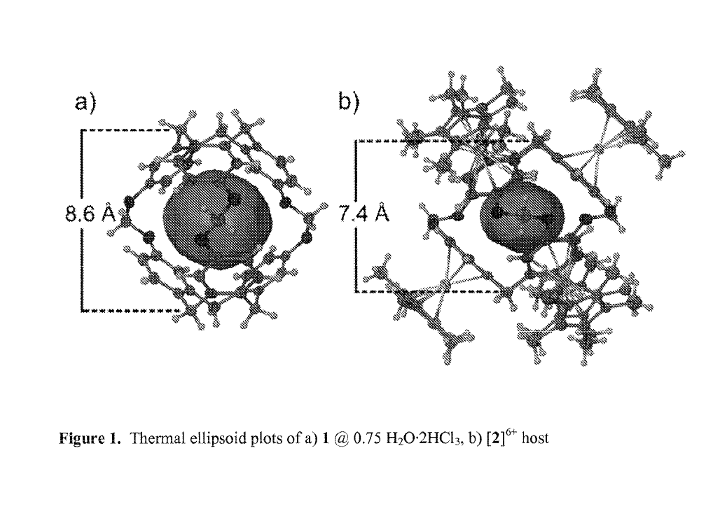

FIG. 2 represents the structure of cryptophane-1,1,1 (1) and synthesis of its permetalated congener [2]Cl₆. i) [Cp*Ru(μ₃-Cl)]₄, H₂O/THF, microwave, 89%.

FIG. 3 shows (A) ¹²⁹Xe NMR spectrum of [2]Cl₆ in D₂O at 293K in the presence of 0.3 bar of Xe gas (host concentration: 1.7 mM). The peak at 308 ppm is assigned to the Xe@[2]⁶⁺ complex and the small peak at 196 ppm is assigned to free, dissolved Xe. (B) Partial plot of the corresponding ¹H spectrum (Hₐ left, Hₑ right; see Chart 1) of [2]Cl₆ in the absence (top) and with 0.3 bar of xenon on top of the solution (bottom).

### DETAILED DESCRIPTION OF' THE INVENTION

Surprisingly, the problems associated with the use of cryptophanes as encapsulating agents have been solved by the present invention which relates to metalated cryptophane derivatives of formula (I):

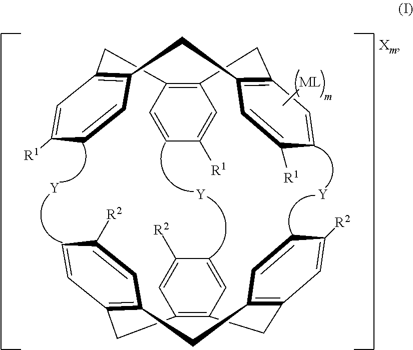

enantiomers and mixtures of enantiomers thereof, wherein

Y is —OZO—; —CH₂ArCH₂—, —CH₂CH≡CHCH₂—, —CH₂C≡CCH₂—, —OCH₂C≡CC≡CCH₂O—, —(OCH₂CH₂)O(OCH₂CH₂)—;

Z═(CH₂)ₙ;

M is transition metal;

L is ligand;

R¹ and R², independently from each other, are H, (C₁-C₃)alkyl, or (C₁-C₃)alkoxy

X is anionic group;

m is an integer from 1 to 6; and

each n is independently 1 or 2.

Another embodiment of the present invention relates to the metalated cryptophane derivatives of formula (I):

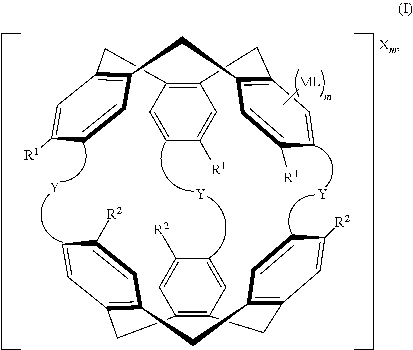

enantiomers and mixtures of enantiomers thereof, wherein

Y is —OZO—; —CH₂ArCH₂—, —CH₂CH═CHCH₂—, —CH₂C≡CCH₂—, —(OCH₂C≡CC≡CCH₂O—, —(OCH₂CH₂)O(OCH₂CH₂)—;

Z═(CH₂)ₙ;

M is transition metal;

L is ligand;

R¹ and R², independently from each other, are H, (C₁-C₃)alkyl, or (C₁-C₃)alkoxy

X is anionic group;

m is an integer from 1 to 6; and

n is 1 or 2.

Another embodiment of the present invention relates to the metalated cryptophane derivatives of formula (I):

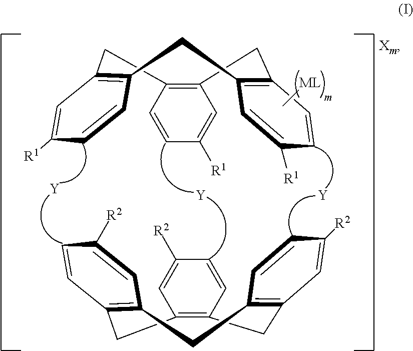

enantiomers and mixtures of enantiomers thereof, wherein

Y is —OZO—;

Z═(CH₂)ₙ;

M is transition metal;

L is ligand;

R¹ and R², independently from each other, are H, (C₁-C₃)alkyl, or (C₁-C₃)alkoxy;

X is halogen, CF₃SO₃⁻ or PF₆⁻;

m is 6; and

each n is independently 1 or 2.

Another embodiment of the present invention relates to the metalated cryptophane derivatives of formula (I):

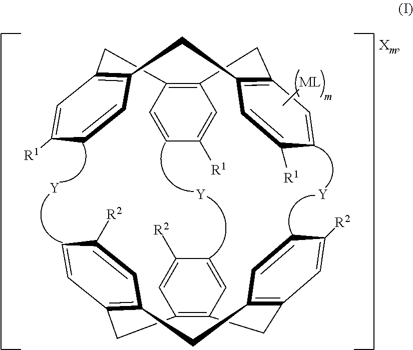

enantiomers and mixtures of enantiomers thereof, wherein

Y is —OZO—;

Z═CH₂)ₙ;

M is transition metal;

L is ligand;

R¹ and R², independently from each other, are H, (C₁-C₃)alkyl, or (C₁-C₃)alkoxy;

X is halogen, CF₃SO₃⁻ or PF₆⁻;

m is 6; and

n is 1 or 2.

Another embodiment of the present invention relates to the metalated cryptophane derivatives of formula (I):

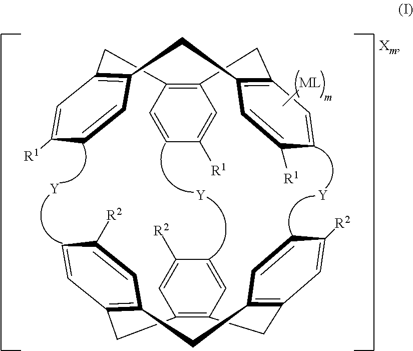

enantiomers and mixtures of enantiomers thereof, wherein

each Y is independently —OCH₂O— or —OCH₂CH₂O—;

M is Ru

L is ligand;

R¹ and R² are H;

X is halogen, CF₃SO₃⁻ or PF₆⁻; and

m is 6.

Another embodiment of the present invention relates to the metalated cryptophane derivatives of formula (I):

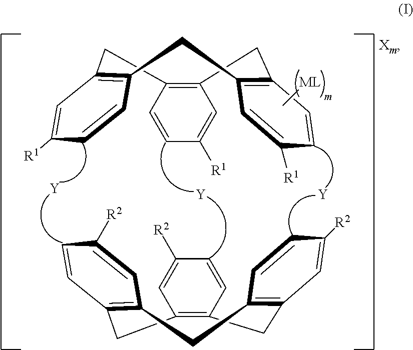

enantiomers and mixtures of enantiomers thereof, wherein

Y is —OCH₂O—;

M is Ru^{II};

L is ligand;

R¹ and R² are H;

X is halogen, CF₃SO₃⁻ or PF₆⁻; and

m is 6.

Another embodiment of the present invention relates to the metalated cryptophane derivatives of formula (I):

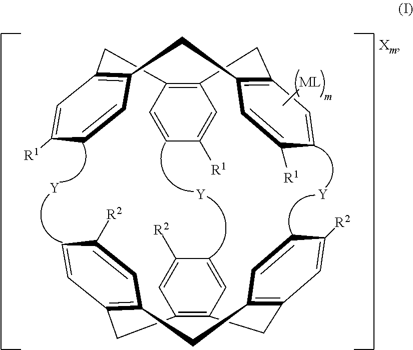

enantiomers and mixtures of enantiomers thereof, wherein

each Y is —OCH₂O— or —OCH₂CH₂O—;

M is Ru^{II};

L is Cp*;

R¹ and R² are H;

X is halogen, CF₃SO₃⁻ or PF₆⁻; and

m is 6.

Another embodiment of the present invention relates to the metalated cryptophane derivatives of formula (I):

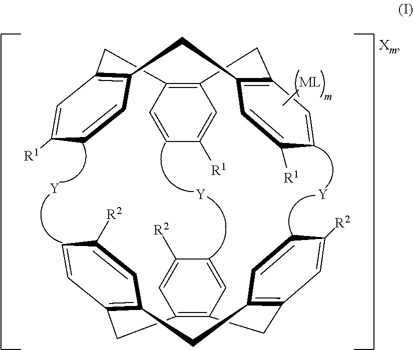

enantiomers and mixtures of enantiomers thereof, wherein

Y is —OCH₂O—;

M is Ru^{II};

L is Cp*;

R¹ and R² are H;

X is halogen, CF₃SO₃⁻ or PF₆⁻; and

m is 6.

Another embodiment of the present invention relates to the metalated cryptophane derivatives of formula (II):

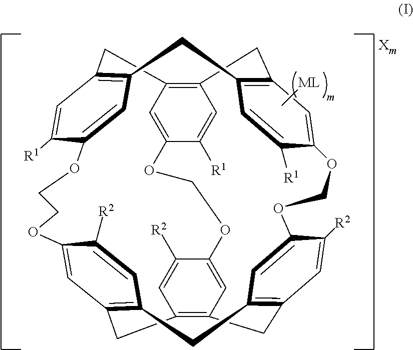

enantiomers and mixtures of enantiomers thereof, wherein

M is transition metal;

L is ligand; R¹ and R², independently from each other, are H, (C₁-C₃)alkyl, or (C₁-C₃)alkoxy

X is anionic group; and

m is an integer from 1 to 6.

Another embodiment of the present invention relates to the metalated cryptophane derivatives of formula (III):

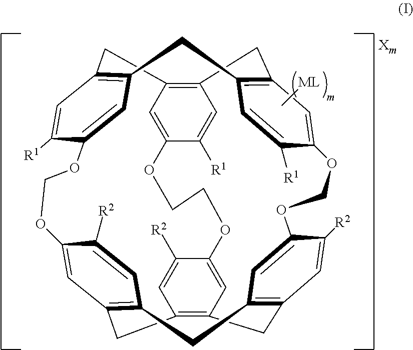

enantiomers and mixtures of enantiomers thereof, wherein

M is transition metal;

L is ligand;

R¹ and R², independently from each other, are H, (C₁-C₃)alkyl, or (C C₃)alkoxy

X is anionic group; and

m is an integer from 1 to 6.

Another embodiment of the present invention relates to the metalated cryptophane derivatives of formula (IV):

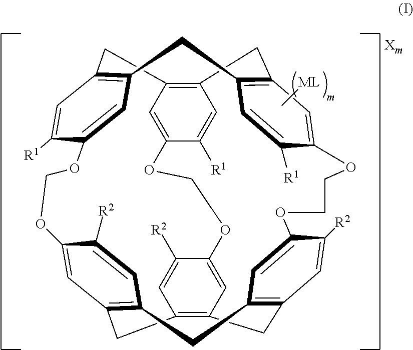

enantiomers and mixtures of enantiomers thereof, wherein

M is transition metal;

L is ligand;

R¹ and R², independently from each other, are H, (C₁-C₃)alkyl, or (C ₁-C₃)alkoxy

X is anionic group; and

m is an integer from 1 to 6.

Another embodiment of the present invention relates to the metalated cryptophane derivatives of formula (V):

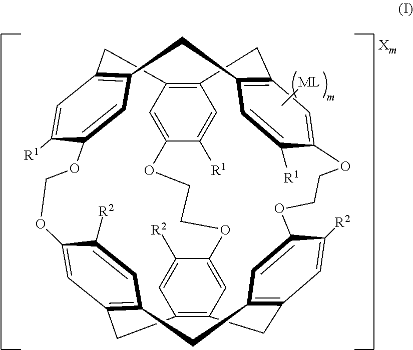

enantiomers and mixtures of enantiomers thereof, wherein

M is transition metal;

L is ligand;

R¹ and R², independently from each other, are H, (C₁-C₃)alkyl, or (C₁-C₃)alkoxy

X is anionic group; and

m is an integer from 1 to 6.

Another embodiment of the present invention relates to the metalated cryptophane derivatives of formula (VI):

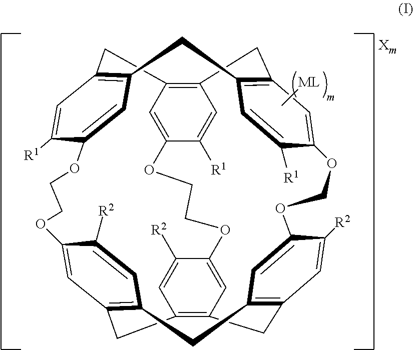

enantiomers and mixtures of enantiomers thereof, wherein

M is transition metal;

L is ligand;

R¹ and R², independently from each other, are H, (C₁-C₃)alkyl, or (C₁-C₃)alkoxy

X is anionic group; and

m is an integer from 1 to 6.

Another embodiment of the present invention relates to the metalated cryptophane derivatives of formula (VII):

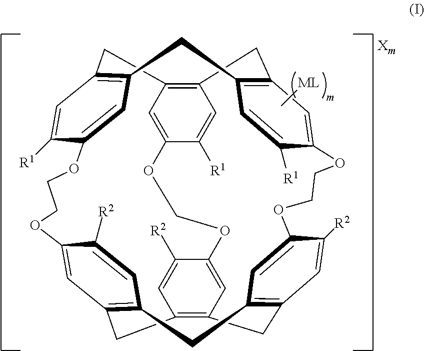

enantiomers and mixtures of enantiomers thereof, wherein

M is transition metal;

L is ligand;

R¹ and R², independently from each other, are H, (C₁-C₃)alkyl, or (C₁-C₃)alkoxy

X is anionic group; and

m is an integer from 1 to 6.

Another embodiment of the present invention relates to the metalated cryptophane derivatives of formula (I), including enantiomers and mixtures thereof, wherein the variables Y,

Z, M, L, R¹, R², X, m and n is any combination wherein:

Y is selected from the group consisting of —OZO ; —CH₂ArCH₂—, CH₂CH═CHCH₂—, —CH₂C≡CCH₂—, —OCH₂C≡CC≡CCH₂O—, and (OCH₂CH₂)O(OCH₂CH₂)—;

Z═(CH₂)ₙ;

M is transition metal;

L is ligand which includes, but is not limited to Cp*;

R¹ and R², independently from each other, are H, (C₁-C₃)alkyl, or (C₁-C₃)alkoxy

X is anionic group, which includes but is not limited to a halogen, CF₃SO₃⁻, PF₆⁻

m is an integer from 1 to 6; and

each n is independently 1 or 2.

Transition metals, as used herein, are the metals, whose atoms have an incomplete d sub-shell, or which can give rise to cations with an incomplete d sub-shell. Such metals include, but are not limited to Fe^{II}, Ru^{II}, Ir^{III} or Rh^{III}. The transition metals represent the transition between group 2 elements and group 13 elements of the Periodic Table. In the d-block the atoms of the elements have between 1 and 10 d electrons. Such transition metals include, but are not limited to Mn, Fe, Co, Ni, Cu, Zn, Mo, Ru, Rh, Pd, Pt, Sc, Zr, and the like.

Transition metals are excellent Lewis acids and accept electron density from many molecules or ions that act as Lewis bases; when a Lewis base donates its electron pair to a Lewis acid, it is said to coordinate to the Lewis acid and form a coordinate covalent bond. When Lewis bases coordinate to metals acting as Lewis acids and form an integral structural unit, a coordination compound is formed. In this type of compound, or complex, the Lewis bases are called ligands.

Ligand within the context of the present invention refers to a molecule, ion or atom that is attached to the central atom of a coordination compound, a chelate or other complex. Ligands, as used herein, can be carbocyclic and heterocyclic aromatic compounds, cyclopentadienyl derivatives (Cp), e.g. Cp*(η⁵-C₅Me₅) or Cp (η₅-C₅H₅), arenes and derivatives thereof, e.g. (ηn⁶-C₆H₆). olefins or polyolefins, macrocycles, such as, for example porphyrin, polydentates, such as, for example biaryls or bipyridines, and the like.

Anionic groups, as used herein, are the common salt-forming anions, which include, but are not limited to halogen (such as, for example Cl⁻), acetate CH₃COO⁻, trifluoroacetate CF₃COO⁻, carbonate CO₃²⁻, citrate HOC(COO⁻)(CH₂COO⁻)₂, phosphates [HₙPO₄]⁽³⁻ⁿ⁾⁻, sulfates [HₙSO₄]⁽²⁻ⁿ⁾⁻, triflate CF₃SO₃⁻, hexafluorophosphate PF₆⁻, hexafluoroantimonate SbF₆⁻, and the like.

Another embodiment of the present invention is a cryptophane of formula (I) which has an empty cavity.

Another embodiment of the present invention relates to the metalated cryptophane derivatives of formula [2]Cl₆, including enantiomers and mixtures of enantiomers thereof:

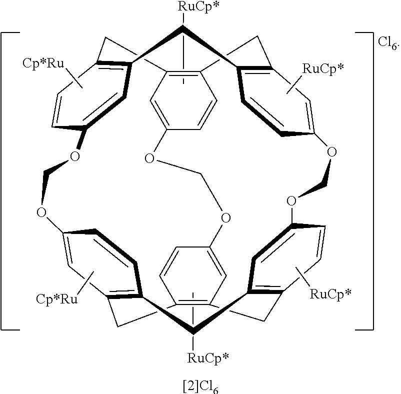

Another embodiment of the present invention relates to the metalated cryptophane derivatives of formula [2]Cl₆ which have an empty cavity (e.g., no small molecules or atoms are within the cavity). See Fairchild et al. J. Am. Chem. Soc., 2010, 132 (44), pp 15505-15507 (incorporated by reference in its entirety herein).

According to another embodiment of the present invention, the water-soluble derivatives of formula (I) can be synthesized by metalation of cryptophane (Ia) with [Cp*Ru(η₃-Cl)]₄ under microwave irradiation or with heating in an appropriate solvent. Alternative methods include the reaction of (Ia) with other appropriate compounds of ruthenium, including, but not limited to, [Cp*Ru(L)ₙ]⁺ compounds, e.g. [Cp*Ru(CH₃CN)ₙ][CF₃SO₃], followed by anion exchange with chloride.

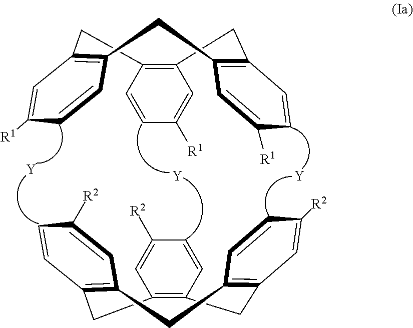

Another embodiment of the present invention is directed to a complex, wherein one or more small molecules or atoms are encapsulated in the spherical cavity of the metalated cryptophane of formula (I).

Another embodiment of the present invention is directed to biosensor complexes of noble gases and cryptophane derivatives of formula (I) according to various embodiments described herein.

Another embodiment of the present invention is directed to xenon biosensor complex with cryptophane of formula [2]Cl₆.

According to another embodiment of the present invention, [2]⁶⁺ corresponds to the host cation.

Another embodiment of the present invention are cryptophanes of formula (I) which are water soluble. In one embodiment, the water solubility of the cryptophanes have a water solubility at standard conditions (25° C. and 1 atm) greater than 100 μM.

Examples of small molecules capable of forming complexes with cryptophanes of formula (I) include, but are not limited to noble gases, such as xenon, radon, krypton; alkanes, such as methane, ethane and the like; haloalkanes; fluorine based compounds such as SF₆; metal cations, such as Na⁺, Cs⁺, Mg²⁻,Ca²⁺, Sr²⁺, Ba²⁺; trivalent lanthanide ions Yb³⁺ and Eu³⁺; ammonium salts; alkyl ammonium cations; tetraalkyl derivatives M(alkyl)₄, wherein alkyl is CH₃ or C₂H₅ and M is Si, Ge, Sn, or Pb; anions such as CF₃SO₃⁻, CF₃CO₂⁻, CH₃SO₃⁻, CH₃CH₂SO₃⁻, SbF₆⁻ and PF₆⁻. Acetylcholine and choline can be also easily encapsulated by cryptophanes.

According to another embodiment, the following isotopes of xenon can be used in the biosensors or in other applications described herein: ¹¹⁰Xe, ¹¹¹Xe, ¹¹²Xe, ¹¹³Xe, ¹¹⁴Xe, ¹¹⁵Xe, ¹¹⁶Xe, ¹¹⁷Xe, ¹¹⁸Xe, ¹¹⁹Xe, ¹²⁰Xe, ¹²¹Xe, ¹²²Xe, ¹²³Xe, ¹²⁴Xe, ¹²⁵Xe, ¹²⁶Xe, ¹²⁷Xe, ¹²⁹Xe, ¹³¹Xe, ¹³²Xe, ¹³³Xe, ¹³⁴Xe, ¹³⁵Xe, ¹³⁶Xe, ¹³⁷Xe, ¹³⁸Xe, ¹³⁹Xe, ¹⁴⁰Xe, ¹⁴¹Xe, ¹⁴²Xe, ¹⁴³Xe, ¹⁴⁴Xe, ¹⁴⁵Xe, ¹⁴⁶Xe,¹⁴⁷Xe or any combination thereof (David R. Lide (ed.), Norman E. Holden in CRC Handbook of Chemistry and Physics, 85th Edition, online version. CRC Press. Boca Raton, Fla. (2005). Section 11, Table of the Isotopes).

Another embodiment of the present invention involves the use of any conformers or conformational isomers of cryptophanes of formula (I). Conformers and conformational isomers are anticipated to exist and are likely to be of great importance for guest encapsulation as conformational changes affect the cavity size and openings that allow substrates to enter and leave the molecular cavity. The conformational modifications are mainly related to the variations of the torsion angles along C—C and C—O bonds of linkers between two orthocyclophane units or within the orthocyclophane units themselves. Cryptophanes of formula (I) possessing orthocyclophane units in any combination of the “out” or “in” cone conformations or “saddle” or “saddle-twist” conformations are possible (Brotin, T.; Dutasta, J.-P. Chem. Rev. 2009, 109, 88-130).

Another embodiment of the present invention involves the use of any regioisomers, stereoisomers or mixtures thereof of crytophanes of formula (I), (II), (III), (IV), (V), (VI) or (VII).

Many compounds of formula (I), (II), (III), (IV), (V), (VI) or (VII) are chiral, and are known to exist in enantiomeric forms. Compounds of the present invention may be present in a single enantiomeric form or as a mixture of enantiomeric forms. In certain embodiments the compound of formula (I), (II), (III), (IV), (V), (VI) or (VII) is present in an enantiomeric excess (ee) of at least 10%, 20%, 30%, 40%, 50%, 60%, 70%, 80%, 90%, 95%, 98% or 99%.

Compounds of formula (I), (II), (III), (IV), (V), (VI) or (VII) may also exist as syn or anti diastereomeric forms, defined by the relative chirality of the two connected cyclotribenzylene units. In some embodiments, the present invention includes syn diastereomers of compounds of formula (I), (II), (III), (IV), (V), (VI) or (VII), enantiomers and mixtures of enantiomers thereof. In some embodiments, the present invention includes mixtures of syn and anti diastereomers of compounds of formula (I), (II), (III), (IV), (V), (VI) or (VII).

Incompletely functionalized cryptophanes of formula (I), (II), (III), (IV), (V), (VI) or (VII) (m=2-4) will exists in various regioisomeric forms, depending upon the relative positions of the ML substitutuents appended to the arene rings of the cryptophane. The present invention includes all regioisomeric forms of formula (I), (II), (III), (IV), (V), (VI) or (VII).

The peripheral substituents of the cryptophanes (such as R¹ and R² of formula (I)) are also important for the dynamics of guest encapsulation, as they influence the size of the portals, allowing access to the molecular cavity. The formation of host-guest complexes is dependent on the accessibility of the molecular cavity, and upon complexation, the conformational populations of the host may change.

The structure and the symmetry of the guest, the electronic density of the aromatic rings of the host, and the solvation effects are also important factors that may influence the formation of the complexes.

Another embodiment of the present invention is directed to a method of using the metalated cryptophane of formula (I) for encapsulation of small molecules, which have to be delivered to the desired biological targets, such as receptors, organs, etc. or removed from the environment as, for example, in case of scavenging pollutants or used to isolate the desired materials from the mixtures or to conduct an isolation process.

Another embodiment of the present invention is directed to the method of using the biosensor complex of a noble gas, e.g. Xe, encapsulated in a cryptophane of formula (I) in clinical imaging.

The noble gas biosensors described herein may be used in any suitable imaging technique known to one of skilled in the art for detecting various diseases or conditions (e.g., cancer, Alzheimer's disease, etc.). Examples of imaging techniques may include, but are not limited to magnetic resonance imaging (MRI), positron emission tomography (PET), and single photon emission computed tomography (SPECT).

In xenon biosensors, laser-polarized xenon atoms are confined inside the specially modified molecular cages. By using optically pumped xenon, the caged-xenon sensor produces much “brighter” signals from chemical targets in living organisms. Most current biosensors use fluorescence, but only a few colors can be used in parallel before the spectra from different biological molecules overlap and obscure one another. The caged-xenon NMR sensors, however, can be “multiplexed” to a high degree, simultaneously using multiple xenon-binding hosts, to detect many distinct analytical targets simultaneously (Berthault, P. et al., Progress in Nuclear Magnetic Resonance Spectroscopy 2009, 55, 35-600)

The principal design conditions required to successfully exploit such biosensors and to easily detect the ¹²⁹Xe NMR signal in vivo are: i) a high binding constant (Kₐ) between xenon and the host molecule in biological media, ii) a large chemical shift difference between bound and free xenon, and iii) adequate xenon in-out exchange, enabling a further gain in sensitivity by constant renewal of the host environment by hyperpolarized xenon.

In one embodiment, the binding constant of the biosensors of the invention is >7,000. In another embodiment, the binding constant is >10,000. In still another embodiment, the binding constant is >20,000. In still another embodiment of the invention the binding constant is within a range selected from the groups consisting of 7,000-100,000; 7,000-50,000; 10,000-100,000; 10,000-50,000; 20,000-100,00; 20,000-50,000 and 25,000-35,000.

For xenon biosensing, the use of the hosts depends of the capacity of grafting on bulky substituents in order to close the cavity and slow down the in-out exchange at the xenon chemical shift time scale.

As mentioned above, an adequate xenon in-out exchange rate is mandatory for the ¹²⁹Xe—NMR based biosensing approach. Obviously, it should be slow on the xenon chemical shift timescale in order to give rise to separate peaks for the caged and free xenon environments, but it should be fast enough to enable constant replenishment of the cage by hyperpolarized xenon. The presence of six [Cp*Ru]⁺ groups on the aromatic rings could conceivably slow down or even stop the in-out xenon exchange

According to another embodiment of the present invention, the metalation of the six arene rings of cryptophane-111 (1) by [Cp*Ru]⁺ moieties results in a cryptophane, [2]Cl₆, that exhibits high water solubility and one of the highest known binding constants for xenon. The cationic, electron withdrawing nature of the [Cp*Ru]⁺ moieties induces an enormous (>275 ppm) downfield chemical shift change for the caged xenon relative to the non-functionalized host. The properties of cryptophanes (I) suggest that it should be possible, using a single, optimized host skeleton (e.g. cryptophane-1,1,1) to design a family of hosts with comparable aqueous xenon affinities, but whose ¹²⁹Xe—NMR frequency responses span nearly the entire known chemical shift range for xenon (0-350 ppm). The approach constitutes an avenue to a family of xenon-optimized biosensors potentially useful for multiplexed imaging applications.

The synthetic conjugation or functionalization of cryptophane hosts through the introduction of various chemical moieties can be used to control/manage the relevant biomolecular properties of the cryptophane structures of formula (I).

The invention is further described by the following non-limiting examples which further illustrate the invention, and are not intended, nor should they be interpreted to, limit the scope of the invention.

### EXAMPLES

### Example 1

### Synthesis of (±)-[(Cp*Ru)₆(1)]Cl₆((±)-[2]Cl₆) and (±)-[Cp*Ru)₆(1)][CF₃SO₃]₆((±)-[²][CF₃SO₃]₆)

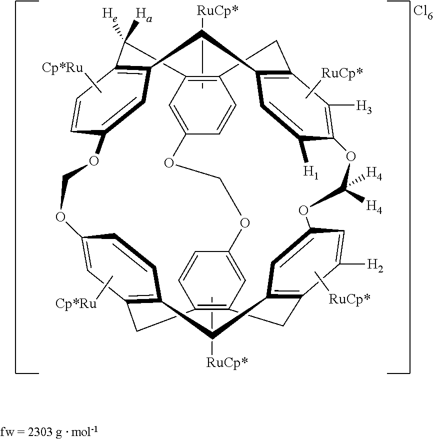

Under an N₂ atmosphere, (±)-cryptophane-111 (1) (30 mg, 0.045 mmol) was dissolved in THF (3 mL) in a 10 mL microwave reaction vessel. [Cp*Ru(μ₃-Cl)]₄ (102 mg, 0.094mmol, 8.4 eq. Ru) was added, followed by degassed water (5 mL). The vessel was sealed and reacted at 130° C. for 30 minutes under microwave irradiation to give a red solution. The solvent of reaction mixture was removed under vacuum.

The solid was added to a silica column and chromatographed using methanol, saturated aqueous NH₄HCO₃, and water (4:4.5:0.5, R_{f}=0.11). The solvent was removed under vacuum at 55° C. To remove excess NH₄HCO₃, additions of methanol/water were added and removed under vacuum at 55° C. stepwise until evolution of NH₃ ceased. The solid was dissolved in H₂O/methanol and passed over Amberlite-IRA 410 Cl beads (chloride ion exchange beads). The solvent was removed under vacuum at 50° C. and the resulting solid was recrystallized from methanol by the addition of diethyl ether to give an off-white powder (yield 91 mg, 89%). Alternatively, the solid can be recrystallized by the diffusion of the vapor into a concentrated aqueous solution of the compound. ¹H NMR (400 MHz, D₂O) δ 6.16 (br. s, 6H, H₁), 6.04 (br. s, 6H, H₃), 5.96 (br. s, 6H, H₂), 5.59 (s, 6H, bridge —OCH₂O—H₄), 3.84 (d, 6H, ²J=13.0 Hz, Hₐ), 2.65 (d, 6H, ²J=13.0 Hz, Hₑ), 1.86 (s, 90H, Cp*); ¹³C NMR (100 MHz, D₂O) δ 127.15, 97.94, 97.72, 97.68, 88.00, 85.38, 76.67, 76.47, 29.89, 9.62. ESI-MS (m/z): calculated for C₁₀₅H₁₂₆O₆Ru₆Cl₄ ([2]≠Cl₄²⁺) 1116.1 found 1115.5; calculated for C₉₅H₁₁₁O₆Ru₅Cl₃ ([(Cp*Ru)₅1].Cl₃²⁺) 980.6, found 980.2; calculated for C₉₅H₁₁₁O₆Ru₅Cl₂ ([(Cp*Ru)₅1].Cl³⁺) 641.8 , found 641.4; calculated for C₈₅H₉₆O₆Ru₄Cl₂ ([(Cp*Ru)₄1].Cl₂²⁺) 844.6 , found 844.1.

Synthesis of [2][CF3SO3]₆: A concentrated, aqueous solution of [NH₄][CF₃SO₃] was added to a concentrated, aqueous solution of [2]Cl₆; the resulting [2][CF₃SO₃]₆ precipitate was collected and dried. Yield was not determined.

### Example 2

### Synthesis of (±)-[((η⁵-C₅Me₅)Ru)₆(1)]Cl₆([Ru₆(1)]Cl₆), C₁₀₅H₁₂₆Ru₆O₆Cl₆, MW=2303.26 g mol⁻¹; [Ru₅(1)]Cl₅, C₉₅H₁₁₁Ru₅O₆Cl₅, MW=2031.51 g mol⁻¹; and [Ru₄ (1)]Cl₄, C₈₅H₉₆Ru₄O₆Cl₄, MW=1759.76 g mol⁻¹; and [Ru₃(1)]Cl₃, C₇₅H₈₁Ru₃O₆Cl₃, MW=1427.96 g mol⁻¹ regioisomeric mixtures:

Under an N₂ atmosphere, cryptophane-111 (1) (59 mg, 0.088 mmol) was dissolved in THF (4 mL) in a 10 mL microwave vessel. [Cp*Ru(η₃-Cl)]₄ (104.9 mg, 0.39 mmol, 4.4 eq.) was added, followed by degassed water (4 mL). The vessel was sealed and reacted at 130° C. for 30 minutes under microwave irradiation to give a transparent, brown solution. The solvent was removed by vacuum.

The resulting solid was dissolved in methanol, spotted onto a silica TLC plate, and chromatographed using a mobile phase of methanol and saturated aqueous NH₄HCO₃ (3:1). Following development, four fractions corresponding to [Ru₆1]Cl₆, [Ru₅1]Cl₅, [Ru₄1]Cl₄, and [Ru₃1]Cl₃ were observed on the TLC plate (R_{f} of [Ru₆1]Cl₆<[Ru₅1]Cl₅<[Ru₄1]Cl₄<[Ru₃1]Cl₃) under UV irradiation. The silica containing each fraction was removed from the TLC plate and was eluted with the mobile phase to give solutions of purified [Ru₆1]Cl₆<[Ru₅1]Cl₅<[Ru₄1]Cl₄<[Ru₃1]Cl₃. The solvents were removed in vacuo (65° C.). Residual NH₄HCO₃ was removed through repeated additions of water/methanol and subsequent removal by rotary evaporation until evolution of NH₃ ceased. The solids were dissolved in water and passed through an Amberlite-IRA 410 Cl beads (for ion exchange). The solvents were removed in vacuo and the resulting solids were recrystallized methanol by addition of diethyl ether to give off-white powders of [Ru₆(1)]Cl₆, [Ru₅(1)]Cl₅, [Ru₄(1)]Cl₄, and [Ru₃(1)]Cl₃.

### Example 3

### Synthesis of (±)-[((η⁵-C₅Me₅)Ru)₃(1)]Cl₃ ([Ru₃(1)]Cl₃), C₇₅H₈₁Ru₃O₆Cl₃, MW=1427.96 g mol⁻¹ [Ru₂(1)]Cl₂, C₆₅H₆₆Ru₂O₆Cl₂, MW=1216.26 g mol⁻¹; and [Ru₁(1)]Cl₁, C₅₅H₅₁Ru₁O₆Cl₁, MW=944.51 g mol⁻¹ regioisomeric mixtures:

Under an N₂ atmosphere, cryptophane-111 (1) (8 mg, 0.012 mmol) was dissolved in THF (4 mL) in a 10 mL microwave vessel. [Cp*Ru(η₃-Cl)]₄ (4 mg, 0.015 mmol, 1.25 eq.) was added, followed by degassed water (4 mL). The vessel was sealed and reacted at 130° C. for 30 minutes under microwave irradiation to give a transparent, colorless solution. The solvent was removed by vacuum. The resulting solid was dissolved in methanol, spotted onto a silica TLC plate, and chromatographed using a mobile phase of methanol, saturated aqueous NH₄HCO₃, and water (5:1:2). Following development, three fractions corresponding to [Ru₃(1)]Cl₃, [Ru₂(1)]Cl₂, and [Ru₁(1)]Cl₁ were observed on the TLC plate (R_{f} of [Ru₃(1)]Cl₃<[Ru₂(1)]Cl₂<1Ru₁(1)]Cl₁) under UV irradiation. The silica containing each fraction was removed from the TLC plate and was eluted with the mobile phase to give solutions of purified [Ru₃(1)]Cl₃, [Ru₂(1)]Cl₂, and [Ru₁(1)]Cl₁. The solvents were removed in vacuo (65° C.). Residual NH₄HCO₃ was removed through repeated additions of water/methanol and subsequent removal by rotary evaporation until evolution of NH₃ ceased. The solids were dissolved in water and passed through an Amberlite-IRA 410 Cl beads (for ion exchange). The solvents were removed in vacuo and the resulting solids were recrystallized methanol by addition of diethyl ether to give off-white powders of [Ru₃(1)]Cl₃, [Ru₂(1)]Cl₂, and [Ru₁(1)]Cl₁.

### Example 4

### Comparison of Xe Bnding Cnstants with other Cyptophane Drivatives

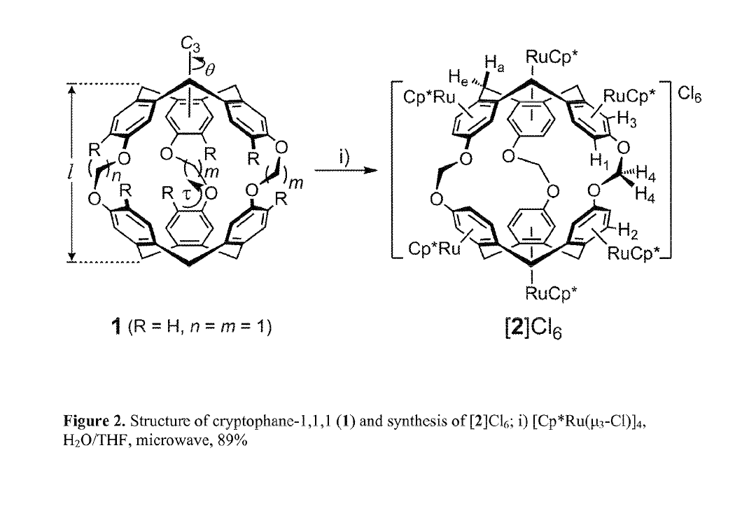

η^{6 -Coordination of the arene rings by cationic, electron-withdrawing [(ηhu 5}-C₅Me₅)Ru^{II]}⁺ moieties (hereafter [Cp*Ru]⁺) gives rise to the water soluble, highly air stable chloride salt [(Cp*Ru)₆1]Cl₆, hereafter [2]Cl₆, (see also FIG. 2) that displays unprecedented affinity for xenon.

The water soluble congeners of other cryptophanes exhibit similar increases in aqueous xenon affinity relative to their organic-soluble parents (Table 1).

TABLE 1
Association constants (293 K) for xenon binding and
¹²⁹Xe chemical shifts of xenon caged in
organic soluble cryptophanes and their water soluble congeners.

| Cryptophane | R¹/R², n, m^{a} | Kₐ | solvent | δe^{b} | ref |
|---|---|---|---|---|---|
| 1 | H, 1, 1 | 10000 | TCE-d₂ | 31 | |
| [2]Cl₆ | H, 1, 1 | 29000 | D₂O | 308 | |
| A (or 222) | OCH₃, 2, 2 | 3300^{d} | TCE-d₂ | 63^{c} | a) |
| A-acid | OCH₂CO₂H, 2, 2 | 6800 | D₂O | 64^{c} | b) |
| 223 | OCH₃, 2, 3 | 2810^{d} | TCE-d₂ | 60 | c) |
| 223-acid | OCH₂CO₂H, 2, 3 | 2200 | D₂O | 52 | b) |
| 233 | OCH₃, 3, 2 | 810^{d} | TCE-d₂ | 47 | c) |
| 233-acid | OCH₂CO₂H, 3, 2 | 2200 | D₂O | 42 | b) |

^{a}See Chart 1.
^{b}Xe@host signal.
^{c}All known derivatives resonate in the ¹²⁹Xe frequency range of 30-80 ppm at room temperature.
^{d}278 K.
^{e}This work.
Ref.:
a) Bartik, K.; Luhmer, M.; Dutasta, J.-P.; Collet, A.; Reisse, J. J. Am. Chem. Soc. 1998, 120, 784-791;
b) Huber, G.; Brotin, T.; Dubois, L.; Desvaux, H.; Dutasta, J.-P.; Berthault, P. J. Am. Chem. Soc. 2006, 128, 6239-6246;
c) Brotin, T.; Dutasta, J. P. Eur. J. Org. Chem. 2003, 973-984.

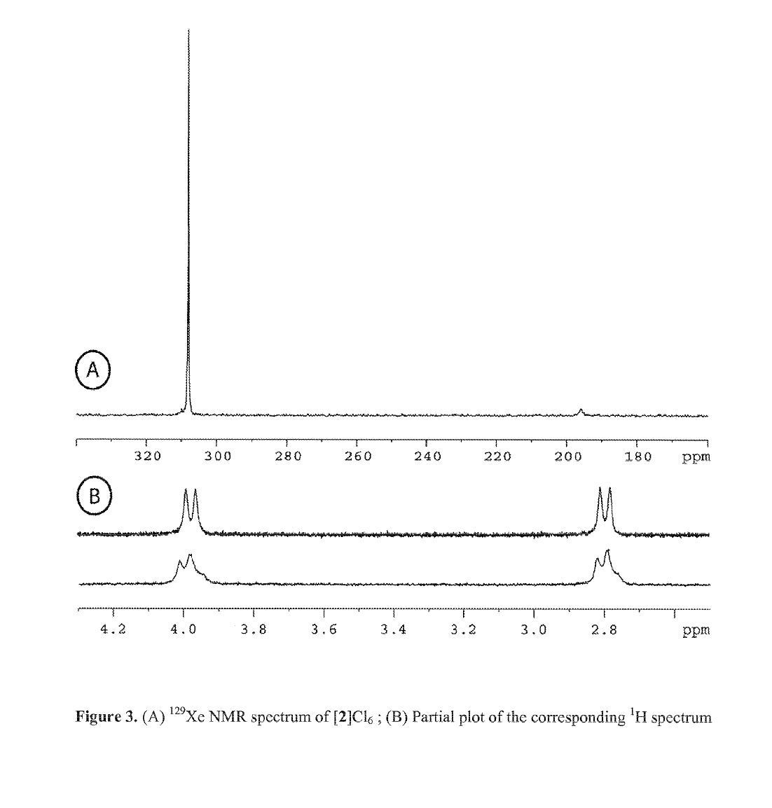

The ¹²⁹Xe NMR spectrum of an aqueous solution of [2]Cl₆ displays two signals, one at 196 ppm for free xenon in water and the other at 308 ppm assigned to the Xe@[2]⁶⁺ complex (FIG. 3). At 293K, slow exchange conditions are encountered both in the ¹H and the ¹²⁹Xe NMR spectra of [2]Cl₆, allowing accurate determination of the binding constant without knowledge of the exact concentration of dissolved xenon. A xenon binding constant of Kₐ=2.9(2)×10⁴ M⁻¹ at 293 K has been extracted. Surprisingly, the xenon affinity of [2]Cl₆ in D₂O is three times higher than that of 1 in TCE-d₂. Indeed, the water soluble congeners of other cryptophanes within the scope of the present invention exhibit similar increases in aqueous xenon affinity relative to their organic-soluble parents (Table 1).

At 308 ppm, the aqueous Xe@[2]⁶⁺ species resonates over 275 ppm downfield from the Xe@1 species in TCE-d₂ (31 ppm). This enormous frequency difference the highest ever observed for two xenon hosts possessing essentially the same internal cavity—is unexpected and is not due to solvent effects. Table 1 gives the resonance frequencies of encapsulated xenon for some organic and water soluble cryptophane congeneric pairs. The encapsulated xenon resonances of the organic-dissolved cryptophanes do not deviate more than a few ppm from the resonances of their water-dissolved congeners (e.g. Xe@A, 63 ppm vs. Xe@A-acid, 64 ppm). Thus, the six cationic, electron-withdrawing [Cp*Ru]⁺ moieties are predominantly responsible for this effect, dramatically affecting the electron density of the caged xenon. Metal functionalization therefore greatly broadens the practical ¹²⁹Xe chemical shift range made available by encapsulation hosts, a feature that augurs well for the development of hosts useful for multiplexed xenon imaging (Berthault, P.; Bogaert-Buchmann, A.; Desvaux, H.; Huber, G.; Boulard, Y. J. Am. Chem. Soc. 2008, 130, 16456-16457).

An adequate xenon in-out exchange rate is mandatory for the ¹²⁹Xe—NMR based biosensing approach. it should be slow on the xenon chemical shift timescale in order to give rise to separate peaks for the caged and free xenon environments, but it should be fast enough to enable constant replenishment of the cage by hyperpolarized xenon. The presence of six [Cp*Ru]⁺ groups on the aromatic rings could conceivably slow down or even stop the in-out xenon exchange. This is not the case, as testified by 2D ¹²⁹Xe EXSY experiments. The extracted exchange rate constants are kₘ=3.8×10⁵ s⁻¹M⁻¹, and kₒᵤₜ=13.1 s⁻¹ at 293 K. These values are consistent with the value of the binding constant considering that free cryptophane is present at 0.011 mM in solution under the 2D ¹²⁹Xe EXSY experimental conditions (1.05 bar xenon).

Having thus described in detail various embodiments of the present invention, it is to be understood that the invention defined by the above paragraphs is not to be limited to particular details set forth in the above description as many apparent variations thereof are possible without departing from the spirit or scope of the present invention.

What is claimed is:

1. Metalated cryptophane derivatives of formula (I): 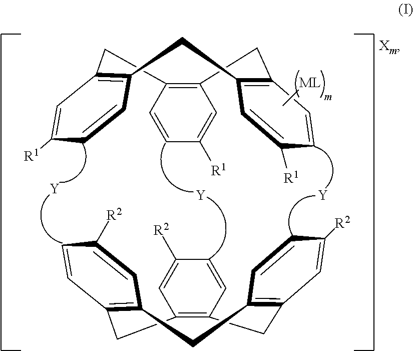
enantiomers and mixtures of enantiomers thereof, wherein
Y is —OZO—; —CH₂ArCH₂—, —CH₂CH═CHCH₂—, —CH₂≡CCH₂—, —OCH₂≡CC═CCH₂O—, —(OCH₂CH₂)O(OCH₂CH₂)—;
Z═(CH₂)ₙ;
M is transition metal;
L is ligand;
R¹ and R², independently from each other, are H, (C₁-C₃)alkyl, or (C₁-C₃)alkoxy X is anionic group;
m is an integer from 1 to 6; and
each n is independently 1 or 2.

2. The metalated cryptophane of claim 1, wherein:
Y is —OZO—;
Z═(CH₂)ₙ;
M is transition metal;
L is ligand;
R¹ and R², independently from each other, are H, (C₁-C₃)alkyl, or (C₁-C₃)alkoxy;
X is halogen, CF₃SO₃⁻ or PF₆⁻;
m is 6; and
each n is independently 1 or 2.

3. The metalated cryptophane of claim 1, wherein:
Y is —OCH₂O—;
M is Ru^{II};
L is ligand;
R¹ and R² are H;
X is halogen, CF₃SO₃⁻ or PF₆⁻; and
m is 6.

4. The metalated cryptophane of claim 1, wherein
Y is —OCH₂O—;
M is Ruⁿ;
L is Cp*;
R¹ and R² are H;
X is halogen, CF₃SO₃⁻ or PF₆⁻; and
m is 6.

5. The metalated cryptophane of claim 1, which has the formula: 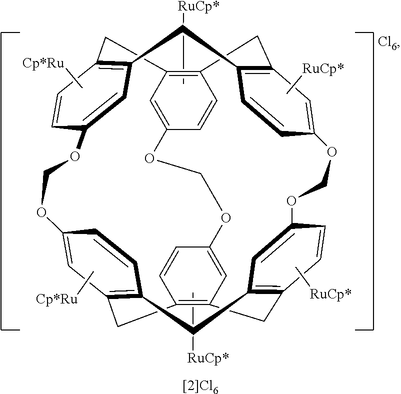
enantiomers and mixtures of enantiomers thereof.

6. A complex which comprises of a small molecule encapsulated in the cavity of the metalated cryptophane of claim 1.

7. The complex of claim 6, wherein the small molecule is a noble gas.

8. The complex of claim 7, wherein the noble gas is xenon and the metalated cryptophane has the formula [2]Cl₆.

9. The complex of claim 8, wherein the xenon is ¹²⁹Xe.

10. A method of detecting a disease or condition in a patient which comprises of applying an imaging technique selected from the group consisting of magnetic resonance imaging (MRI), positron emission tomography (PET), and single photon emission computed tomography (SPECT) to a patient being diagnosed wherein the imaging technique uses the biosensor complex of claim 6.

11. The method of claim 10, wherein the imaging technique is magnetic resonance imaging (MRI).

12. The method of claim 11, wherein the biosensor complex comprises xenon as the noble gas and the metalated cryptophane has the formula [2]Cl₆

13. The method of claim 12, wherein the xenon is ¹²⁹Xe.

14. The transition metal modification(s) of arene rings of cryptophane-A, cryptophane-111 (1) or their derivatives defined by formula II (n≦2) so as to affect the Nuclear Magnetic Resonance chemical shift or binding affinity of encapsulated species, such as, but not limited to, xenon nuclei.

15. A process of making the metalated cryptophane derivatives of claim 1 which comprises metalating a cryptophane of formula (1a): 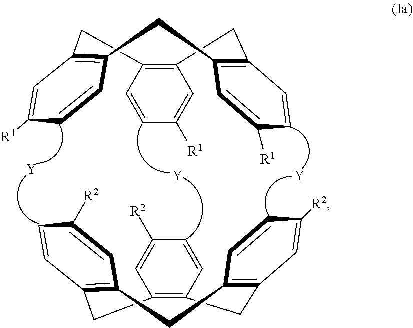
enantiomers and mixtures of enantiomers thereof, wherein with heat and in the presence of a solvent, when R¹, R² and Y are as defined in claim 1.
